(19) United States
(12) Patent Application Publication
(10) Pub. No.: US 2026/0124466 A1
(43) Pub. Date: May 7, 2026

(54) WEARABLE LIGHT THERAPY DEVICE WITH INTEGRATED VIBRATION ELEMENT

(71) Applicant: Eduardo Marti, Weston, FL (US)

(72) Inventor: Eduardo Marti, Weston, FL (US)

(21) Appl. No.: 19/253,600

(22) Filed: Jun. 27, 2025

Related U.S. Application Data
(63) Continuation-in-part of application No. 18/935,470, filed on Nov. 2, 2024.

(51) Int. Cl. A61N 5/06; A61H 23/02

(52) U.S. Cl. CPC A61N 5/0624; A61H 23/02; A61H 2201/10; A61H 2201/165; A61N 2005/0626; A61N 2005/0645; A61N 2005/0652; A61N 2005/0659; A61N 2005/0663

(57) ABSTRACT

A wearable radiation emitting device with integrated vibration element which operates to provide for the use of radiation treatment in conjunction with vibration treatment. The wearable radiation emitting device with integrated vibration element includes a plurality of LEDs, some of which emit blue visible light and others which emit red visible light and infrared light, and are arranged to direct these emissions to an injury site so as to reduce general pain as well as promote a wearer's recovery by way of increased blood flow and infection prevention. The wearable radiation emitting device with integrated vibration element also includes a plurality of integrated vibrating pads operative to generate vibration around an injury site that also promotes increased blood flow as well as stimulates the lymphatic system, all of which promotes systemic healing and aids in infection prevention and swelling reduction.

### CROSS REFERENCE TO RELATED APPLICATIONS

This application is a continuation in part of, incorporates by reference, and claims the benefit of co-pending U.S. nonprovisional patent application Ser. No. 18/935,470, filed Nov. 2, 2024.

### BACKGROUND OF THE INVENTION

### Field of the Invention

This invention relates generally to wearable therapeutic devices and, more particularly, to a wearable device which provides for the application of therapeutic radiation at a plurality of wavelengths and vibration to a targeted part of a wearer's body.

### Description of the Prior Art

Injuries which result in soft tissue damage, or even incisions or an amputation due to trauma or surgery, often require post injury or post operative treatment. Such treatments often take on various forms, and may include a combination of rehabilitation, pain management, infection prevention and repair of the skin at or around the site of the injury. Such treatment has been shown to improve the recovery success rate for the injury and promote the surgical site to heal correctly without secondary issues.

While there are various treatment methods for soft tissue damage, amputees and general incision recovery, it has been found that light therapy, such as therapy in the visible light and infrared spectrums, can provide a range of benefits with relative ease of application. Such light therapy is commonly provided through light emitting diode (“LED”) devices. Indeed, the application of various specific spectrums of visible and infrared light has been shown to promote blood flow and oxygenation to the site and therefore increase the rate at which the site heals while reducing scarring. Light spectrums such as near infrared blue light have also been shown to reduce bacterial infection by killing unwanted bacteria such as Staphylococcus virus, which is a major risk factor in post-op scenarios.

Vibration therapy can also be used as a treatment method for soft tissue damage, amputees and general incision recovery. Vibration has been shown to dilate blood vessels and improve blood flow to the site as well as stimulate the lymphatic system. Stimulating the lymphatic system has been shown to reduce the risk of infection and promote systemic healing. As such, the stimulation of blood flow and lymphatic stimulation in various recovery scenarios through vibration has been found to greatly improve recovery.

A key benefit of improved healing is that it can oftentimes reduce the amount of pain experienced by the patient. The effects of the various infrared light spectrums also reduce the likelihood of conditions such as phantom limb pain syndrome, which is often triggered by blood flow issues to the amputation site, excessive swelling or infection.

Accordingly, there remains a need for a radiation emitting device which provides light therapy by emitting radiation at wavelengths which are specifically for treating soft tissue damage as well as amputation and incision sites, and which has an integrated vibration element, such as medical vibrating pads, for lymphatic system stimulation. It would be desirable for such a radiation emitting device with integrated vibration element to utilize LEDs which emit near spectrum red and blue light, as well as infrared light, all at wavelengths effective for use in treating various skin and limb issues. It would additionally be desirable for the vibrating pad(s) of such a radiation emitting device with integrated vibration element to be programmable to provide vibration stimulation in periods of stimulation and rest, which has been found to be most effective.

With such a device, both light and vibration therapies can be applied to all the above-mentioned conditions at the same time, only requiring the use of a single device. Advantageously, this would allow this type of treatment to be suitable for the patient to conduct themselves or with minimal assistance at various stages of the rehabilitation process.

### SUMMARY OF THE INVENTION

The present disclosure provides for a wearable radiation emitting device with integrated vibration element, comprising: a device body having a first side and an opposing second side, wherein the device body is adapted to be positioned on a wearer's body adjacent to a target area on the wearers body; a plurality of electrical radiation emitting aspects positioned on the first side and configured to selectively generate radiation when activated, wherein the plurality of radiation emitting aspects are arranged such that when activated with the device body adjacent to the target area, the plurality of radiation emitting aspects direct radiation into the target area; and at least one vibrating element adapted to selectively produce vibrational motion, wherein the at least one vibrating element is integrated with the device body such that such that when activated with the device body adjacent to the target area, motion produced by the at least one vibrating element is transferred the wearer's body

It is an object of the present disclosure to provide a radiation emitting device which provides light therapy by emitting radiation at wavelengths which are specifically for treating soft tissue damage as well as amputation and incision sites, and which has an integrated vibration element, such as medical vibrating pads, for lymphatic system stimulation.

It is an additional object of the present disclosure to provide a radiation emitting device with integrated vibration element which utilizes LEDs which emit near spectrum red and blue light, as well as infrared light, all at wavelengths effective for use in treating various skin and limb issues.

It is an additional object of the present disclosure to provide a radiation emitting device with integrated vibration element with a vibrating pad(s) which is programmable to provide vibration stimulation in periods of stimulation and rest, as has been found to be most effective.

### BRIEF DESCRIPTION OF THE DRAWINGS

FIG. 1 is a plan view of a wearable radiation emitting device with integrated vibration element built in accordance with the present disclosure, showing a control side of a device body with a detachable controller.

FIG. 2 is a plan view of a wearable radiation emitting device with integrated vibration element built in accordance with the present disclosure, showing the control side of the device body with the detachable controller removed.

FIG. 3 is a plan view of the detachable controller of a wearable radiation emitting device with integrated vibration element built in accordance with the present disclosure, showing a first side thereof.

FIG. 4 is a plan view of a wearable radiation emitting device with integrated vibration element built in accordance with the present disclosure, showing the treatment side of the device body.

FIG. 5 is a top plan view of a removable cover of a wearable radiation emitting device with integrated vibration element built in accordance with the present disclosure.

FIG. 6 is a graph showing an exemplary single wavelength output for LEDs in a wearable radiation emitting device with integrated vibration element built in accordance with the present disclosure.

FIG. 7 is a graph showing an exemplary multiple wavelength output for LEDs in a wearable radiation emitting device with integrated vibration element built in accordance with the present disclosure.

### DETAILED DESCRIPTION OF THE INVENTION

Described herein is a wearable radiation emitting device with integrated vibration element which operates to provide for the use of radiation treatment in conjunction with vibration treatment. This type of device may be used to assist the rehabilitation and healing process, as well as for pain management for patients with various conditions, mostly relating to soft tissue damage, post operative treatment, amputation or general incisions, or skin damage due to scarring and infection. The wearable radiation emitting device with integrated vibration element disclosed herein has been shown to deliver benefits which include healing soft tissue injuries faster while helping to prevent secondary infections, as well as improve the quality of skin repair in the affected area and reducing scarring and swelling.

The wearable radiation emitting device with integrated vibration element enables the use of various spectrums of light, such as infrared and visible light, to promote healing through increased blood flow and oxygenation to an affected area. Advantageously, light spectrums may be selected which also assist in killing bacteria such as staphylococcus aureus, and other harmful microbes. Because infections can often disrupt the healing and rehabilitation progress when dealing with post op scenarios that involve an incision site, the ability to kill harmful microbes provides a significant benefit.

In addition to the infrared and other light spectrums, the wearable radiation emitting device with integrated vibration element also utilizes vibration to further increase blood flow to the trauma site by dilating blood vessels which promotes faster healing. This is particularly important for tendons and ligaments, which naturally have very little blood flow in the area. The vibration also stimulates the lymphatic system, which further assists with preventing infection and leads to systemic healing, as well as reduces swelling by regulating fluid buildup in the region.

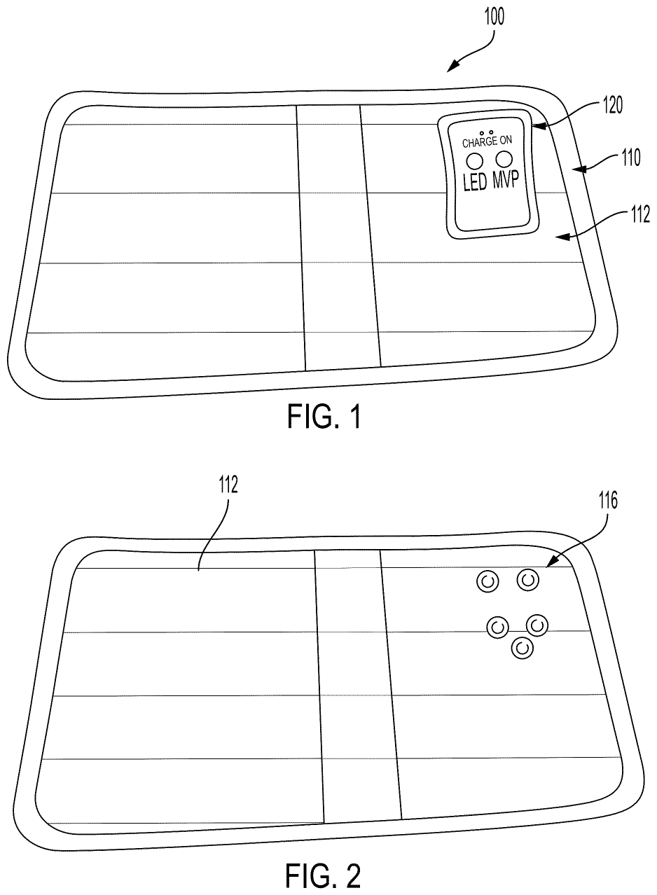

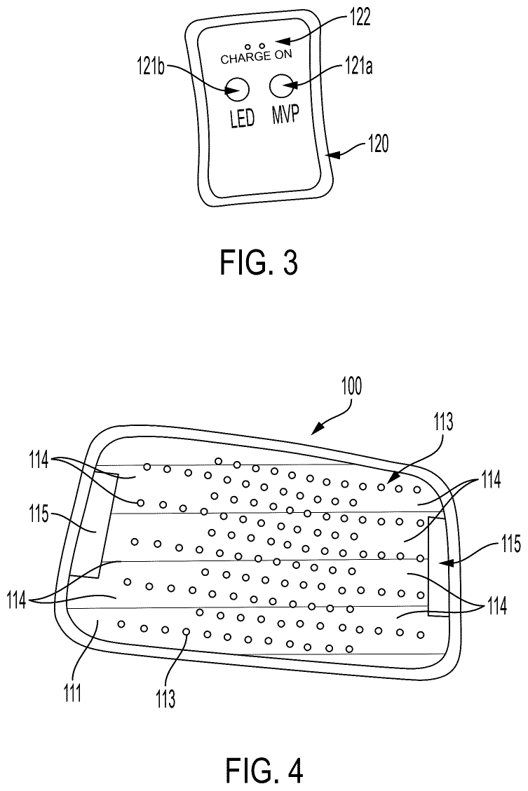

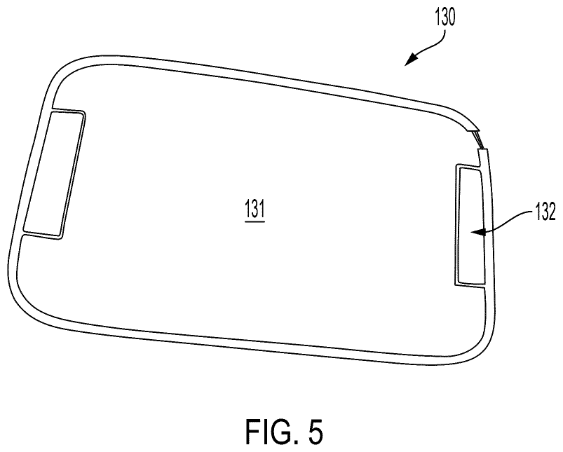

Referring now to the drawings and, in particular, FIGS. 1, 2, 3, 4 and 5, a wearable radiation emitting device with integrated vibration element 100 is shown as a brace body 110 and a control module 120. The brace body 110 may be defined by a substantially flat member, such as a wearable wrap or soft brace which can be fitted to an area of treatment, having a treatment side 111 and an opposing control side 112, and may be constructed of a nylon, foam, neoprene and breathable mesh material. The brace body 110 may be edged with binding to protect the material from wear while preventing any unfinished edges from scratching against a wearer.

The treatment side 111 of the brace body 110 includes a plurality of LED lights 113 mounted to its surface and a plurality of vibrating pads 114 which are positioned right below the outer layer of the surface of the treatment side 111. The LED lights 113 may be deployed in multiple groups, with each group having both infrared, red (visible) and blue (visible) LEDs in alternating rows to allow for full coverage of all required spectrums. In this regard, when the brace body 110 is fitted on a wearer, these LEDs may be orientated so that they are positioned to direct radiation in the region of the injury site so as to be suitable to provide light therapy.

The treatment side 111 may also include a pair of fastener sections 115 which allow for attachment of a protective cover 130. The fastener sections 115 may be formed of either a hook portion of a hook and loop fastener or the loop portion of a hook and loop fastener.

The control side 112 of the brace body 110 includes magnetic mounting interface 116, which allows for the removable attachment of the control module 120 to the brace body 110 and thus may be exposed when the control module 120 is not attached to the brace body 110. The magnetic mounting interface 113 may be constructed of or include a plurality of connectors constructed of a ferromagnetic material, arranged in a pattern such as what is shown in FIG. 2, and may be electrically connected to the LED lights 113 and the vibrating pads 114. As such, the magnetic mounting interface 113, together with a magnetic coupler of the control module 120, allow for the control module 120 to be removably attached magnetically to the brace body 110 and for the transmission of electricity and electrical signals between the control module 120 and the LED lights 113 and the vibrating pads 114.

In some embodiments, magnetic mounting interface 113 may also include, or be constructed as, a mechanical clip, such as a spring biased clip.

The control module 120 may be constructed of rigid housing made of injected plastic and may include therein a controller and a power source such as a rechargeable or replaceable battery, and include thereon a magnetic coupling interface (not shown), with each of these items electrically interconnected such that the controller can use power from the power source to generate electrical signals and send electrical signals it has generated through the magnetic coupler. The magnetic coupling interface may be formed of a plurality of coupling members constructed of a ferromagnetic material and arranged in a pattern that corresponds to the magnetic mounting interface 113 shown in FIG. 2. The housing may feature a minimalistic and sleek design to allow for comfortable use and wear, with rounded edges preventing any scratching or digging into a wearer.

The control module 120 includes a pair of buttons 121a, 121b or other manual actuators, with each of the buttons 121a, 121b electrically connected to the controller and being designated to control the operation of either the LED lights 113 on the brace body 110 or the control the operation of the vibrating pads 114 in the brace body 110. These buttons 121a, 121b may be labeled on the control module 120 according to their function for easy use.

The control module 120 may also include charge indicator lights 122 which are electrically connected to the controller and used to allow the controller to visually indicate whether battery level is low or sufficiently charged. These lights may be positioned near the top of the control module 120.

The battery in the control module 120 serves as the power source for the electrical components of the wearable radiation emitting device with integrated vibration element 100. Advantageously, having to disconnect the control module 120 for battery charging or replacement ensures that the device is not used during such times for safety purposes. Also, the brace body 110 is arranged such with the control module 120 on the flip side as the treatment side 111 to prevent cases where fluid can come in contact with the control module 120 while the wearable radiation emitting device with integrated vibration element 100 is being worn. Moreover, this allows for the control module 120 to remain easily accessible and charge indicator lights visible when the wearable radiation emitting device with integrated vibration element 100 is in place on a wearer.

The LED lights 113 on the treatment side 111 may be grouped into defined treatment regions, with the placement of treatment regions varying depending on the specific injury or body part being treated. For a brace body 110 that is suited for a wearer's back, such as the brace body 110 illustrated in FIGS. 1, 2, and 4, the treatment regions may include an intense area with densely clustered LEDs focused on the lower spine and erector muscles of the lower back. The treatment regions may also include moderate area where LEDs are less densely clustered on the sides of and moving further away from the intense area. This operates to ensure that the area being treated is not only the immediate region of the injury site but the area around the injury site so as to provide a more holistic treatment for the wearer and further reduce any issues surrounding the injury site. Such an approach can assist greatly with reducing overall inflammation and swelling.

The LED lights 113 may be made up of a first group of LED bulbs which emit a first type of radiation and a second group of LED bulbs which emit radiation at one or more other spectrums. For example, first group of LED bulbs may emit a 470 nm wavelength blue light in the visible spectrum, while the second group of LED bulbs may emit infrared radiation and the second group may emit radiation a 660 nm red light in the visible spectrum, a 830 nm infrared light, and a 950 nm infrared light. The first group and the second group of LEDs may be placed in an alternating pattern so that the various wavelengths of light are evenly distributed to the treatment region.

In the illustrated embodiment, the vibrating pads 114 may be positioned around the intense area treatment region, interspersed with the moderate area treatment region. This allows the vibrating pads 114 to be located on either side of the spine, as well as on the sides of the waist. This also causes the vibrating pads 114 to be evenly spaced to give complete coverage to the area being treated, and to affect the lymphatic system in the area being treated as well as adjacent areas.

The brace body 110 may also be used with a protective cover 130, with the protective cover 130 being formed from a clear plastic screen having binding 131 on the edges to prevent scratching a wearer and allow for a uniform finish. The protective cover 130 may operate to prevent fluid or leakage from contaminating the device when used on a wearer with open wounds or on a recently operated region. The protective cover 130 may include protector fastener 132 defined by a portion of a hook and loop fastener which corresponds to the portion of the hook and loop fastener which define the fastener sections 115, so as to allow for the removable attachment of the protective cover 130 to the brace body 110. In this regard, not only can the protective cover 130 protect the LED lights 113 and the vibrating pads 114, it also can be removed for cleaning in between uses.

It is appreciated that if a wearer's treatment site has no open wound, then the brace body 110 may be worn without the protective cover 130.

It is contemplated that the wearable radiation emitting device with integrated vibration element 100 may be deployed as either a standalone device, or as an add-on to an existing brace. In the case of the wearable radiation emitting device with integrated vibration element 100 being used on its own, it may be provided with conventional adjustable mounting straps (not shown) to allow for universal fitment. In cases where the device is used in conjunction with another brace, the wearable radiation emitting device with integrated vibration element 100 would be able to attach thereto via an elastic loop 117 centrally located thereon. In this regard, the wearable radiation emitting device with integrated vibration element 100 may provide either for universal adjustment or be sized to fit, so as to be mountable on wearers of different sizes or on different body parts of a wearer. At the same time, the wearable radiation emitting device with integrated vibration element 100 may be structured to allow for sufficient airflow to prevent excess perspiration and heat build-up.

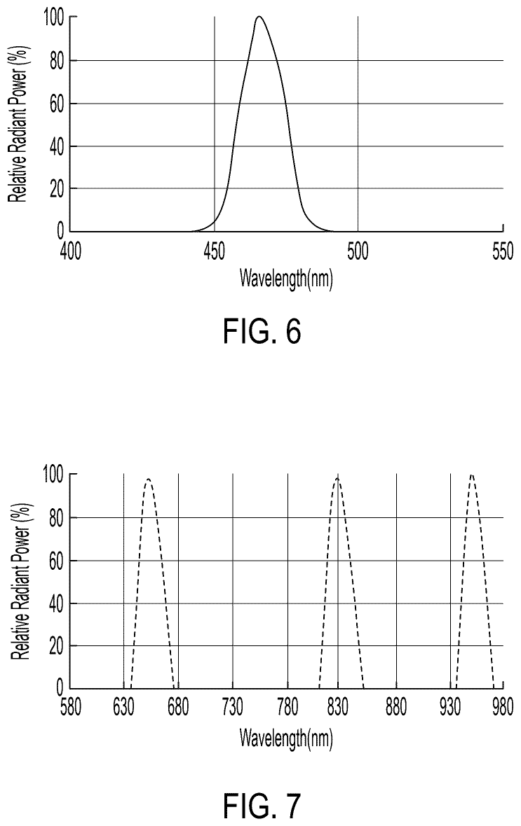

Referring now to FIG. 6, the wavelength emitted by the 470 nm LED light when operating at full relative radiant power is indicated. As is evident, the wavelength varies from around 450 nm to 490 nm as the light is operated. When operating at full capacity as it would be on the wearable radiation emitting therapy device, it emits light in the 470 nm wavelength spectrum.

Referring now to FIG. 7, the three-in-one LED light emits a spectrum around three target wavelengths. The exact wavelengths are achieved when the LED light is operating at full relative radiant power, as shown. These wavelengths are 660 nm, 830 nm, and 950 nm, respectively. It is contemplated, however, that these wavelengths may vary by roughly 20 nm in either direction as the LED lights reach their full power, where the exact frequency is achieved.

It is appreciated that the LED light therapy, particularly using a variety of wavelengths of light, assists in various ways, such as promoting blood circulation, which in turn improves oxygenation. It also assists in detoxification and an influx of antibodies to the region. Other benefits include improved scar healing and general skin healing through increased collagen production. Furthermore, as certain wavelengths have antibacterial properties, such that the blue light emits, these assists in killing bacteria including staphylococcus and other skin bacteria.

For example, the infrared light is able to penetrate deep into the skin and tissue and promote blood flow and therefore provide improved oxygenation as well as a greater influx of antibodies to the site. This phenomenon, in conjunction with the increased collagen production, results in improved skin repair and cellular regeneration, boosts new cell growth, enhances skin rejuvenation and stimulates various cellular processes and increases rejuvenation.

The blue light has similar benefits to infrared light with regards to increasing blood flow and its associated benefits. In addition, blue LED light therapy stimulates fibroblasts, the cells responsible for collagen production in the skin. This can help reduce the appearance of fine lines, wrinkles, and even scars. Advantageously, blue light has also been shown to have the most effective antimicrobial spectral range. This assists in killing various bacteria and fungi, allowing the skin to heal without secondary ailments such as fungal infection, acne and infections such as staphylococcus.Blue light has also been shown to suppress cells that contribute to inflammation in the skin, which can also treat chronic inflammatory skin conditions such as eczema.

The instant disclosure employs vibration therapy synergistically with the radiation treatment. The vibrating pads, which are used to provide for the vibration therapy, may be placed in such a way that they are in locations which will supplement the radiation treatment by stimulating the lymphatic system and increasing blood flow in and around the treatment area. These pads may be set to vibrate at a frequency which optimizes the stimulation of the lymphatic system and the increase in blood flow, such as at 13000 RPM, +/−3000, with a twenty-five (25) second on and five (5) second off cycle.

It is appreciated that the lymphatic system assists in defending the body against infection, which is important to keep trauma sites healthy during recovery. Stimulating the lymphatic system can also lead to systemic healing. The lymphatic system is also responsible for regulating fluid levels in the body. This is especially important when dealing with soft tissue damage as patients often experience a fluid buildup at the trauma site when dealing with soft tissue injuries. As such, stimulating the lymphatic system in these regions will assist in reducing the swelling by transporting the excess fluid away from the injury site. While doing so it will also filter out waste or abnormal cells from this fluid, helping to keep the body healthy.

The instant invention has been shown and described herein in what is considered to be the most practical and preferred embodiment. It is recognized, however, that departures may be made therefrom within the scope of the invention and that obvious modifications will occur to a person skilled in the art.

1. A wearable radiation emitting device with integrated vibration element, comprising:
a device body having a first side and an opposing second side, wherein the device body includes a left edge and a right edge and the device body is adapted to be positioned on a wearer's body adjacent to a target area on the wearer's body;
a plurality of radiation emitting aspects positioned on the first side and configured to selectively generate radiation when activated, wherein the plurality of radiation emitting aspects are arranged such that when activated with the device body adjacent to the target area, the plurality of radiation emitting aspects direct radiation into the target area;
a plurality of vibrating elements, each adapted to selectively produce vibrational motion, wherein the plurality of vibrating elements are integrated with the device body such that when activated with the device body adjacent to the target area, motion produced by the plurality of vibrating elements is transferred to the wearer's body; and
wherein the plurality of vibrating elements are positioned on the device body such that a left vibrating element among the plurality of vibrating elements is proximate to the left edge and a right vibrating element among the plurality of vibrating elements is proximate to the right edge, thereby enabling motion produced by the plurality vibrating elements to be transferred to at least the target area when activated.

2. The wearable radiation emitting device with integrated vibration element of claim 1, wherein the plurality of radiation emitting aspects are configured to selectively generate radiation at a plurality of wavelengths when activated.

3. The wearable radiation emitting device with integrated vibration element of claim 1, wherein the plurality of radiation emitting aspects include at least a first region of radiation emitting aspects, a second region of radiation emitting aspects, and a third region of radiation emitting aspects, with the first region of radiation emitting aspects being more densely clustered than both the second region of radiation emitting aspects and the third region of radiation emitting aspects.

4. The wearable radiation emitting device with integrated vibration element of claim 3, wherein the first region of radiation emitting aspects, the second region of radiation emitting aspects, and the third region of radiation emitting aspects each include at least one discrete radiation emitting aspect which generates radiation at a first wavelength when activated and at least one discrete radiation emitting aspect which generates radiation at a second wavelength when activated.

5. The wearable radiation emitting device with integrated vibration element of claim 1, additionally comprising a control module operative to selectively activate the radiation emitting aspects and the plurality of vibrating elements, wherein the control module is removably attached to the second side and the control module and device body are configured such that electrical operating power is only supplied to the radiation emitting aspects and the plurality of vibrating elements when the control module is attached to the second side.

6. The wearable radiation emitting device with integrated vibration element of claim 1, wherein the plurality of radiation emitting aspects are defined by a set of LED lights.

7.-8. (canceled)

9. The wearable radiation emitting device with integrated vibration element of claim 1, additionally comprising a protective cover sized to be placed on and completely cover the first side and be removably attached to the first side.

10. A wearable radiation emitting device with integrated vibration element, comprising:
a device body having a first side and an opposing second side, wherein the device body includes a left edge and a right edge and the device body is adapted to be positioned on a wearer's body adjacent to a target area on the wearer's body;
a plurality of radiation emitting aspects positioned on the first side and configured to selectively generate radiation when activated, wherein the plurality of radiation emitting aspects are arranged such that when activated with the device body adjacent to the target area, the plurality of radiation emitting aspects direct radiation into the target area;
wherein the plurality of radiation emitting aspects include at least a first region of radiation emitting aspects, a second region of radiation emitting aspects, and a third region of radiation emitting aspects, with the first region of radiation emitting aspects being more densely clustered than both the second region of radiation emitting aspects and the third region of radiation emitting aspects;
a plurality of vibrating elements, each adapted to selectively produce vibrational motion, wherein the plurality of vibrating elements are integrated with the device body such that when activated with the device body adjacent to the target area, motion produced by the plurality of vibrating elements is transferred to the wearer's body; and
a control module operative to removably attach to the device body and selectively activate the radiation emitting aspects and the plurality of vibrating elements, wherein the control module and device body are configured such that electrical operating power is only supplied to the radiation emitting aspects and the plurality of vibrating elements when the control module is attached to the device body.

11. The wearable radiation emitting device with integrated vibration element of claim 10, wherein the plurality of radiation emitting aspects are defined by at least a first discrete set of LED lights and a second discrete set of LED lights, with the first discrete set of LED lights being operative to selectively generate radiation at a first wavelength when activated and the second discrete set of LED lights being operative to selectively generate radiation at at least one wavelength that is not the first wavelength when activated.

12. The wearable radiation emitting device with integrated vibration element of claim 11, wherein:
the first discrete set of LED lights and the second discrete set of LED lights are positioned in an alternating pattern in the first region of radiation emitting aspects, the second region of radiation emitting aspects, and the third region of radiation emitting aspects.

13. (canceled)

14. The wearable radiation emitting device with integrated vibration element of claim 10, wherein the plurality of vibrating elements are positioned on the device body such that a left vibrating element among the plurality of vibrating elements is proximate to the left edge and a right vibrating element among the plurality of vibrating elements is proximate to the right edge, thereby enabling motion produced by the plurality vibrating elements to be transferred to at least the target area when activated.

15. The wearable radiation emitting device with integrated vibration element of claim 10, wherein the second region of radiation emitting aspects is proximate to the left edge, the third region of radiation emitting aspects is proximate to the right edge, and the first region of radiation emitting aspects is flanked by both the second region of radiation emitting aspects and the third region of radiation emitting aspects, thereby enabling the plurality of radiation emitting aspects to be arranged such that when activated with the device body adjacent to the target area, the plurality of radiation emitting aspects direct radiation into secondary areas adjacent to the target area in addition to the target area.

16. The wearable radiation emitting device with integrated vibration element of claim 10, additionally comprising a protective cover sized to be placed on and completely cover the first side and be removably attached to the first side.

17. A wearable radiation emitting device with integrated vibration element, comprising:
a device body having a first side and an opposing second side, wherein the device body includes a left edge and a right edge and the device body is adapted to be positioned on a wearer's body adjacent to a target area on the wearer's body;
a plurality of radiation emitting aspects positioned on the first side and configured to selectively generate radiation when activated, wherein the plurality of radiation emitting aspects are arranged such that when activated with the device body adjacent to the target area, the plurality of radiation emitting aspects direct radiation into the target area;
wherein the plurality of radiation emitting aspects include at least a first region of radiation emitting aspects, a second region of radiation emitting aspects, and a third region of radiation emitting aspects, with the first region of radiation emitting aspects being more densely clustered than the second region of radiation emitting aspects and the third region of radiation emitting aspects;
a plurality of vibrating elements, each adapted to selectively produce vibrational motion, wherein the plurality of vibrating elements are is integrated with the device body, positioned on the device body such that a left vibrating element among the plurality of vibrating elements is proximate to the left edge and a right vibrating element among the plurality of vibrating elements is proximate to the right edge, thereby enabling motion produced by the plurality of vibrating elements to be transferred to the at least the target area; and
a control module operative to removably attach to the second side and selectively activate the radiation emitting aspects and the plurality of vibrating elements, wherein the control module and device body are configured such that electrical operating power is only supplied to the radiation emitting aspects and the plurality of vibrating elements when the control module is attached to the second side.

18. The wearable radiation emitting device with integrated vibration element of claim 17, wherein the plurality of radiation emitting aspects are defined by at least a first discrete set of LED lights and a second discrete set of LED lights, with the first discrete set of LED lights being operative to selectively generate radiation at a first wavelength when activated and the second discrete set of LED lights being operative to selectively generate radiation at least one wavelength that is not the first wavelength when activated.

19. The wearable radiation emitting device with integrated vibration element of claim 18, wherein the first discrete set of LED lights and the second discrete set of LED lights are positioned in an alternating pattern in the first region of radiation emitting aspects, the second region of radiation emitting aspects, and the third region of radiation emitting aspects.

20. The wearable radiation emitting device with integrated vibration element of claim 19, additionally comprising a protective cover sized to be placed on and completely cover the first side and be removably attached to the first side.

21. The wearable radiation emitting device with integrated vibration element of claim 3, wherein the second region of radiation emitting aspects is proximate to the left edge and the third region of radiation emitting aspects is proximate to the right edge.

22. The wearable radiation emitting device with integrated vibration element of claim 21, wherein the first region of radiation emitting aspects is flanked by both the second region of radiation emitting aspects and the third region of radiation emitting aspects, thereby enabling the plurality of radiation emitting aspects to be arranged such that when activated with the device body adjacent to the target area, the plurality of radiation emitting aspects direct radiation secondary areas adjacent to the target area in addition to the target area.

23. The wearable radiation emitting device with integrated vibration element of claim 17, wherein the first region of radiation emitting aspects is flanked by both the second region of radiation emitting aspects and the third region of radiation emitting aspects, thereby enabling the plurality of radiation emitting aspects to be arranged such that when activated with the device body adjacent to the target area, the plurality of radiation emitting aspects direct radiation secondary areas adjacent to the target area in addition to the target area.
